(19) United States
(12) Patent Application Publication
(10) Pub. No.: US 2019/0106682 A1
(43) Pub. Date: Apr. 11, 2019

(54) Live, Attenuated Alphavirus Constructs and Methods and Uses Thereof

(71) Applicant: Takeda Vaccines, Inc., Cambridge, MA (US)

(72) Inventors: Jill Ann Livengood, Fort Collins, CO (US); Timothy Duane Powell, Maynard, MA (US)

(73) Assignee: Takeda Vaccines, Inc., Cambridge, MA (US)

(21) Appl. No.: 16/088,815

(22) PCT Filed: Mar. 28, 2017
(86) PCT No.: PCT/US2017/024450

§ 371 Date: Sep. 26, 2018

Related U.S. Application Data
(60) Provisional application No. 62/316,264, filed on Mar. 31, 2016.

(51) Int. Cl. C12N 7/00; A61K 39/12

(52) U.S. Cl. CPC C12N 7/00; A61K 39/12; A61K 2039/5254; C12N 2770/36134; C12N 2770/36162; C12N 2770/36121

(57) ABSTRACT

Embodiments herein relate to live, attenuated alphaviruses that are incapable of replicating in mosquito cells and of transmission by mosquito vectors. Other embodiments concern methods of generating live, attenuated alphaviruses, constructs thereof and uses of these live, attenuated alphaviruses in immunogenic compositions. Other embodiments relate to pharmaceutical compositions including the live, attenuated alphaviruses and methods for manufacturing these live, attenuated viruses. Yet other embodiments relate to uses of these compositions in kits for portable applications such as vaccines against alphavirus infection and methods thereof.

### PRIORITY

This PCT application claims the benefit under 35 USC § 119(e) of provisional U.S. patent application Ser. No. 62/316,264 filed on Mar. 31, 2016, which is incorporated herein by reference in its entirety.

### FIELD OF INVENTION

Embodiments herein relate to live, attenuated alphaviruses that are incapable of replicating in mosquito cells and incapable of transmission by mosquito vectors. Other embodiments concern methods of generating live, attenuated alphaviruses and constructs thereof. In yet other embodiments, live, attenuated alphaviruses disclosed herein can be of use to generate an immunogenic composition against infection by the alphavirus. Other embodiments relate to pharmaceutical compositions including the live, attenuated alphaviruses and methods for manufacturing these live, attenuated viruses. Some embodiments relate to uses of these compositions in kits for portable applications such as immunogenic compositions against alphavirus infection and methods thereof. Certain embodiments disclosed herein concern recombinant alphavirus constructs.

### BACKGROUND

Vaccines to protect against pathogenic virus infections have been effectively used to reduce the incidence of human disease. One of the most successful technologies used for viral vaccines is to immunize animals or humans with a weakened or attenuated strain of the virus (a “live, attenuated virus”). Due to limited replication after immunization, the attenuated strain does not cause disease in the host or other subject because it has been modified. However, this limited viral replication is sufficient to express a full repertoire of viral antigens and can generate potent and long-lasting immune responses to the virus in a subject receiving such a vaccine. Therefore, upon subsequent exposure to a pathogenic strain of the virus, the immunized individual can be protected from disease caused by the virus. These live, attenuated viral vaccines are amongst the most successful vaccines used in public health.

The Alphavirus genus in the Togaviridae family contains a number of significant human and animal pathogens. These viruses are widely distributed on all continents except for the Antarctic region, and represent a significant public health threat. Most of the alphaviruses are transmitted by mosquitoes, in which they can cause a life-long infection in the host. In vertebrates infected by mosquitoes during their blood meal, alphaviruses can cause an acute infection, characterized by a viremia that is a prerequisite of infection of new mosquitoes and its continued circulation in nature. Vaccines against these viruses are limited.

There are health concerns that genetically altered viruses of use in vaccine formulations can be introduced into the natural circulation, mediated by mosquito vectors, and may demonstrate further evolution during long-term replication, either in mosquitoes or during viremia development in vertebrate hosts. Therefore, in designing a new generations of live virus strains of use in vaccines, it is important to create attenuated viruses that are incapable of being spread by their vector such as a mosquito.

### SUMMARY

Embodiments herein relate to live, attenuated alphaviruses that are incapable of replicating in mosquito cells and of transmission by mosquito vectors. Other embodiments concern methods for generating live, attenuated alphaviruses, constructs thereof and uses of these live, attenuated alphaviruses in immunogenic compositions. Other embodiments relate to pharmaceutical compositions that can include the live, attenuated alphaviruses disclosed herein and methods for manufacturing these pharmaceutical compositions. Yet other embodiments relate to uses of these immunogenic compositions against alphaviruses in kits for portable applications and methods.

In some embodiments, live, attenuated, recombinant alphaviruses can include an alphavirus nucleic acid, having an insertion of an internal ribosomal entry site of at least one virus between one end of nonstructural protein 4 (nsP4) coding sequence and initiating AUG of a subgenomic RNA coding sequence of the alphavirus. In accordance with these embodiments, an internal ribosome entry site (IRES) sequence can include different IRES sequences inserted into the genome of the alphavirus which enables transcription/expression of the genes in mammalian cells. These IRES sequences are in contrast, essentially incapable of expression in insect cells. In certain embodiments, different IRES sequences can include, but are not limited to, one or more IRES sequences from picornaviruses designated as Type I (e.g. enterovirus: subtype, enterovirus 71 (EV71) and subtype, human rhinovirus (HRV)) and/or one or more IRES sequences from picornaviruses designated as Type II IRES sequences (e.g., cardiovirus: subtype, theilovirus (TMEV) other than EMCV IRES, and aphthovirus: subtype, foot-and-mouth disease virus (FMDV)). In other embodiments, an IRES sequence can include one or more IRES sequences derived or found in flaviviruses (e.g., subtype, Hepacivirus (HCV)).

In another embodiment, live, attenuated recombinant alphaviruses disclosed herein can include an inactivated subgenomic promoter in addition to insertion of an IRES sequence as disclosed above. In accordance with these embodiments, an inactivated, subgenomic promoter can be inactivated by any method known in the art such as mutagenesis, point mutations, deletions and insertions, (e.g. that do not result in a frameshift or where certain regions of translation are conserved) and the like. In other embodiments, an alphavirus subgenomic promoter can be inactivated by a deletion of the 5′ UTR of the subgenomic RNA between the end of nonstructural protein 4 (nsP4) coding sequence and initiating AUG of a subgenomic RNA coding sequence. In other embodiments, an alphavirus subgenomic promoter of the live, attenuated recombinant alphaviruses can be inactivated by clustered point mutations in the alphavirus nucleic acid located in the 5′UTR of the subgenomic RNA. In other embodiments, live, attenuated recombinant alphaviruses disclosed herein can include one or more mutations in the subgenomic promoter wherein the mutations do not modify the amino acid sequence of the carboxy terminus of nonstructural protein 4. In yet other embodiments, the live, attenuated recombinant alphaviruses can further include adaptive mutations in any one of the non-structural proteins of the alphavirus, wherein the adaptive mutations increase virus replication, release and virus titers. In other embodiments, the live, attenuated viruses can include manipulations for codon optimization in order to optimize translation of the live attenuated viruses.

In certain embodiments, live, attenuated recombinant alphaviruses of the present invention can include live, attenuated recombinant alphaviruses incapable of replicating in mosquitoes and mosquito cells. In accordance with these embodiments, the live, attenuated recombinant alphaviruses are capable of expression in mammals and mammalian cells.

In other embodiments, live attenuated alphaviruses and alphavirus constructs can include, but are not limited to, alphaviruses such as chikungunya virus, o'nyong'nyong virus, Ross River virus, Eastern equine encephalitis virus, Venezuelan equine encephalitis virus, Western equine encephalitis virus or other alphaviruses in the Coronaviridae and Togaviridae families. Other Semliki Forest virus complexes include, but are not limited to, Bebaru virus, Mayaro virus, Subtype: Una virus, O'Nyong Nyong virus: Subtype: Igbo-Ora virus, Ross River virus: Subtype: Bebaru virus; Subtype: Getah virus; Subtype: Sagiyama virus, Semliki Forest virus: Subtype: Me Tri virus. In certain embodiments, the live attenuated alphavirus and/or alphavirus construct can be a live, attenuated chikungunya virus.

In certain embodiments, vectors can include a nucleotide sequence encoding a live, attenuated, recombinant alphavirus disclosed herein. Some embodiments can include a host cell capable of expressing the vector having the nucleotide sequence encoding the live, attenuated, recombinant alphaviruses.

In some embodiments, a live, attenuated recombinant alphavirus or alphavirus constructs disclosed herein can be part of a pharmaceutical composition and can include a pharmaceutically acceptable carrier. In accordance with these embodiments, live, attenuated recombinant alphavirus or constructs disclosed herein as part of a pharmaceutical composition can be administered to a subject in order to induce an immune response to the live, attenuated recombinant alphavirus or alphavirus constructs. In certain embodiment, pharmaceutical compositions disclosed herein can be part of a vaccine composition of use to administer in one or more doses to a subject. In accordance with these embodiments, a subject can be a human subject (e.g. adult or child or infant) or livestock such as a horse, cattle, camels, alpacas or other domestic animal or pet.

In certain embodiments, immunogenic alphavirus compositions disclosed herein can be mixtures of polypeptides and polynucleotides of the alphavirus. In accordance with these embodiments, immunogenic alphavirus compositions can include polynucleotides described herein with or without various polypeptides encoded by polynucleotides. In other embodiments, methods disclosed herein can include administering one or more doses of a pharmaceutically acceptable immunogenic composition of the live, attenuated alphaviruses to a subject by any acceptable method, including for example, subcutaneously, intramuscularly, intravenously, intradermally, transdermally, orally, via inhalation, intravaginally, topically, intranasally or rectally or other known application. In accordance with these embodiments, immunogenic formulations can be administered in a single dose or in two or more doses to a subject. In some embodiments, immunogenic formulations can be administered within about 365 days (1 year) of each other or less, within about 120 days of each other or less, within about 90 days or less of each other, within about 60 days or less of each other, within about 30 days or less of each other, and within less than about 30 days of each other. In other embodiments, two or more doses can be provided to a subject on the same day at the same or different anatomical locations. In yet other embodiments, the live, attenuated alphavirus constructs can be mixed with other vaccines compatible for administration to a subject in a single or multiple doses, for example, administered to travelers entering endemic areas infested with mosquitos capable of carrying pathogenic viruses.

In other embodiment, live, attenuated recombinant alphaviruses, constructs and compositions disclosed herein can be part of a kit including, but not limited to at least one live, attenuated, recombinant alphavirus or construct disclosed herein and at least one container. In yet other embodiments, kit can further include, but are not limited to, one or more stabilizing agents, saline or buffer, and/or delivery device. It is noted that any of the above referenced embodiments can be combined in order to achieve goals by the applicant.

### BRIEF DESCRIPTION OF THE DRAWINGS

The following drawings form part of the instant specification and are included to further demonstrate certain aspects of particular embodiments herein. The embodiments may be better understood by reference to one or more of these drawings in combination with the detailed description presented herein.

FIG. 1 illustrates a schematic method of generating a live, attenuated, recombinant alphavirus of some embodiments of the present invention.

FIG. 2 illustrates alternative schematic methods of generating a live, attenuated, recombinant alphavirus of some embodiments of the present invention.

FIG. 3 represents a graphic illustration of certain live, attenuated alphaviruses of some embodiments of the present invention grown in mammalian cells compared to a positive and negative control.

FIG. 4 represents a graphic illustration of certain live, attenuated alphaviruses of some embodiments of the present invention grown in insect cells.

FIG. 5 illustrates plaque morphology of certain live, attenuated alphaviruses of some embodiments of the present invention when compared to a positive control.

FIG. 6 is a bar graph illustrating neutralization titers for certain live, attenuated alphaviruses of some embodiments of the present invention compared to investigational vaccine and a positive control of a live, attenuated alphavirus.

FIG. 7 is a table illustrating neutralization titer ranges of certain live, attenuated alphaviruses of some embodiments of the present invention compared to a positive control live, attenuated alphavirus and an investigational vaccine.

FIG. 8 is a table illustrating expected neutralizing titer ranges following challenge with wild type alphavirus following administration of pharmaceutical compositions of various live, attenuated alphavirus construct of some embodiments of the present invention compared to a positive control alphavirus constructs.

FIG. 9 illustrates an exemplary alignment of several alphaviruses having an IRES insertion.

### BRIEF DESCRIPTION OF THE SEQUENCES

SEQ ID NO:1 is an example of a polynucleotide sequence of a CHIKV-EV71 IRES; SEQ ID NO:2 is an example of a polynucleotide sequence of a CHIKV-HRV2 IRES; SEQ ID NO:3 is an example of a polynucleotide sequence of a CHIKV-FMDV IRES; SEQ ID NO:4 is an example of a polynucleotide sequence of a CHIKV-TMEV IRES; SEQ ID NO:5 is an example of a polynucleotide sequence of a CHIKV-HCV IRES; SEQ ID NO:6 is an example of a polynucleotide sequence of a EV71 IRES; SEQ ID NO:7 is an example of a polynucleotide sequence of a HRV2 IRES; SEQ ID NO:8 is an example of a polynucleotide sequence of a FMDV IRES; SEQ ID NO:9 is an example of a polynucleotide sequence of a TMEV IRES; SEQ ID NO:10 is an example of a polynucleotide sequence of a HCV IRES; SEQ ID NO:11 is an example of a polynucleotide sequence of a CHIKV-HAV IRES; SEQ ID NO:12 is an example of a polynucleotide sequence of a CHIKV-FGF1 IRES; SEQ ID NO:13 is an example of a polynucleotide sequence of a HAV IRES; SEQ ID NO:14 is an example of a polynucleotide sequence of a FGF1 IRES; SEQ ID NO:15 is an example of a DNA sequence of a primer of use in certain embodiments disclosed herein; SEQ ID NO:16 is an example of a DNA sequence of a primer of use in certain embodiments disclosed herein; SEQ ID NO:17 is an example of a DNA sequence of a probe of use in certain embodiments disclosed herein; SEQ ID NO:18 represents some structural polypeptides of a CHIK-IRES; SEQ ID NO:19 represents non-structural polypeptides of a CHIK-IRES non-structural polypeptide and SEQ ID NO:20 is an example of a polynucleotide sequence of an EMCV-IRES.

### DESCRIPTION OF ILLUSTRATIVE EMBODIMENTS

### Definitions

As used herein, “a” or “an” may mean one or more than one of an item.

As used herein, “about” may mean up to and including plus or minus five percent, for example, about 100 may mean 95 and up to 105.

As used herein, “attenuated virus” can mean a virus that demonstrates reduced or no clinical signs of disease when administered to an animal.

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some cases, well-known methods or components have not been included in the description.

In accordance with embodiments disclosed herein, there can be employed conventional molecular biology, protein chemistry, microbiology, and recombinant DNA techniques within the skill of the art. Such techniques are explained fully in the literature. See for example, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Certain embodiments disclosed herein relate to novel constructs of live, attenuated alphaviruses. In accordance with these embodiments, these live, attenuated alphaviruses are capable of replicating efficiently in vertebrate cells, but have reduced to no expression in insect cells. In some embodiments, live, attenuated, recombinant alphaviruses can include an alphavirus nucleic acid, having an insertion of an internal ribosomal entry site (IRES) of at least one virus between one end of nonstructural protein 4 (nsP4) coding sequence and initiating AUG of a subgenomic RNA coding sequence of the alphavirus. In accordance with these embodiments, an IRES sequence can include different IRES sequences capable of creating a construct that drives expression of genes in mammalian cells. In certain embodiments, different IRES sequences can include, but are not limited to, one or more IRES sequences from picornaviruses designated as Type I (e.g. enterovirus: subtype, enterovirus 71 (EV71) and subtype, human rhinovirus (HRV)) and/or one or more IRES sequences from picornaviruses designated as Type II IRES sequences other than EMCV IRES, (e.g., cardiovirus: subtype, theilovirus (TMEV) and aphthovirus: subtype, foot-and-mouth disease virus (FMDV)). In other embodiments, an IRES sequence can include one or more IRES sequences derived or found in flaviviruses (e.g., subtype, Hepacivirus (HCV)). It is noted that the constructs disclosed herein do not have an EMCV IRES insertion.

In other embodiments, live, attenuated recombinant alphaviruses disclosed herein can include an inactivated subgenomic promoter. In accordance with these embodiments, a live, attenuated recombinant alphavirus can include an IRES insertion as detailed herein and further include an inactivated subgenomic promoter by mutation or other form of inactivation of an alphavirus of interest (e.g. CHIK). In some embodiments, a live, attenuated recombinant alphavirus disclosed herein can be viable and can be characterized as a highly attenuated phenotype while still capable of replicating and inducing an immune response against the alphavirus in a subject when administered for example, to reduce or prevent alphavirus infection in the subject. In accordance with these embodiments, an inactivated, subgenomic promoter can be inactivated by any method known in the art such as mutagenesis, point mutations, deletions, insertions (e.g. that do not result in a frameshift or where certain regions of translation are conserved) and the like. In other embodiments, the subgenomic promoter of the live, attenuated recombinant alphaviruses can be inactivated by clustered point mutations in the alphavirus nucleic acid located in the 5′UTR of the subgenomic RNA. In other embodiments, live, attenuated recombinant alphaviruses disclosed herein can include one or more mutations in the subgenomic promoter wherein the mutations do not modify the amino acid sequence of the carboxy terminus of nonstructural protein 4. In yet other embodiments, the live, attenuated recombinant alphaviruses can further include adaptive mutations in any one of the non-structural proteins of the alphavirus, wherein the adaptive mutation can increase virus replication, virus release and/or virus titers, for example, for manufacturing purposes.

In certain embodiments, attenuation of alphaviruses by passaging either in tissue culture or in embryos can lead to point mutations in structural and nonstructural genes of the alphavirus, and in the cis-acting elements of viral genomes. In addition, mutations can be increased by chemical mutagenesis. In some embodiments, genetic manipulations by altering the sequences of infectious cDNA clones of the RNA+ viruses can be used to create stable and significant modification of viral genomes (e.g. deletions, insertions, reversions, mutations), that would make it very difficult to or impossible to revert to the wild-type or other insect-infectious genome sequence, or additional genetic material that might enhance the immunogenicity of the variants. In certain embodiments, live, attenuated recombinant alphaviruses of the present invention can include live, attenuated recombinant alphaviruses incapable of replicating in mosquitoes and mosquito cells. In accordance with these embodiments, the live, attenuated recombinant alphaviruses are capable of expression in mammals and mammalian cells.

In some embodiments, live, attenuated alphaviruses and alphavirus constructs can include, but are not limited to, alphaviruses such as chikungunya virus, o'nyong'nyong virus, Ross River virus, Eastern Equine Encephalitis Virus, Venezuelan Equine Encephalitis Virus, Western Equine Encephalitis virus or other alphaviruses in the Coronaviridae and Togaviridae families. Other Semliki Forest virus complexes include, but are not limited to, Bebaru virus, Mayaro virus, Subtype: Una virus, O'Nyong Nyong virus: Subtype: Igbo-Ora virus, Ross River virus: Subtype: Bebaru virus; Subtype: Getah virus; Subtype: Sagiyama virus, Semliki Forest virus: Subtype: Me Tri virus. In certain embodiments, the live attenuated alphavirus and/or alphavirus construct is Chikungunya virus. In other embodiments, a pharmaceutical composition against pathogenic alphaviruses can include multiple live, attenuated alphaviruses or other live, attenuated or killed viruses in a compatible combination pharmaceutical composition for administration to a subject.

Alphaviruses are small, spherical, enveloped, positive-sense RNA viruses responsible for a considerable number of human and animal diseases including disease in livestock. For example, chikungunya virus (CHIKV; Togaviridae: Alphavirus) is a arboviral pathogen that has recently caused explosive urban outbreaks involving millions of people across the world such as in Africa and Asia. Live arbovirus vaccine strains against these viruses should not be transmissible by arthropod vectors, because circulation among reservoir hosts could lead to unforeseen changes that might include increased virulence and at least a chance of transmission to another host. The former risk was underscored by the detection of the Venezuelan equine encephalitis virus (VEEV) TC-83 vaccine strain in mosquitoes collected in Louisiana during 1971, an area outside the epizootic/epidemic that was restricted to Texas. This vaccines strain, VEEV TC-83, is capable of replicating in mosquito cells, and infecting mosquitoes following vaccination; therefore, its transmission by mosquitoes remains a possibility.

In certain embodiments, vectors for expressing a nucleotide sequence disclosed herein are contemplated of use to generate live, attenuated alphaviruses or constructs disclosed herein. In other embodiments, vectors can be used for expressing nucleotide sequences encoding live, attenuated, recombinant alphaviruses or alphavirus construct disclosed herein. Some embodiments can include a host cell capable of expressing the vector having the nucleotide sequence encoding the live, attenuated, recombinant alphaviruses. It is contemplated that any vector capable of expressing these constructs may be of use for methods disclosed herein.

In other embodiments, a live, attenuated recombinant alphavirus or one or more alphavirus constructs disclosed herein can be part of a pharmaceutical composition and can include a pharmaceutically acceptable carrier. In accordance with these embodiments, live, attenuated recombinant alphavirus or constructs disclosed herein forming part of a pharmaceutical composition can be administered to a subject in order to induce an immune response to the live, attenuated recombinant alphavirus or alphavirus constructs. In certain embodiments, pharmaceutical compositions disclosed herein can be part of a vaccine composition of use to administer in one or more doses to a subject. In accordance with these embodiments, a subject can be a human subject (e.g. adult or child or infant) or livestock or other animals such as a horse, cattle, camels, alpacas, zoo or wild animals or domesticated animal or pet.

In certain embodiments, immunogenic alphavirus compositions disclosed herein can be mixtures of polypeptides and polynucleotides. In accordance with these embodiments, immunogenic alphavirus composition can include polynucleotides described herein with or without various polypeptides encoded by polynucleotides. In other embodiments, methods can include administering one or more doses of a pharmaceutically acceptable immunogenic composition disclosed herein to a subject by any known mode. In accordance with these embodiments, modes of administration can include, but are not limited to, subcutaneously, intramuscularly, intravenously, intradermally, transdermally, orally, via inhalation, intravaginally, topically, intranasally or rectally administering the composition to a subject. In accordance with these embodiments, immunogenic formulations can be administered in a single dose or in two or more doses to a subject. In some embodiments, immunogenic formulations can be administered within about 365 days (1 year) of each other or less, about 120 days of each other or less, within about 90 days or less of each other, within about 60 days or less of each other, within about 30 days or less of each other, and within less than about 30 days of each other as a booster. In certain embodiments, a subject can be an adult, a child or an infant or animal. In other embodiments, immunogenic compositions disclosed herein can be administered subcutaneously or intradermally or intranasally to a subject on one day and subsequently administered a second dose, boost, on another day within 30 days or up to 6 months to one year after the first dose.

Currently, few vaccines or therapeutic agents against chikungunya are available, so outbreaks of these viruses can only be controlled by preventing the exposure of people to mosquito vectors and treating the symptoms of disease. During the 1980s, scientists at the Walter Reed Army Institute of Research produced an investigational vaccine called 181/clone 25 (hereafter referred to as the 181-25). This live, attenuated strain was generated through serial plaque-to-plaque passages of a wild-type CHIKV strain. The virus is attenuated in both rodents and non-human primates and is highly immunogenic in humans. However, this 181-25 strain caused mild, transient arthralgia and was found to be transmitted experimentally by the natural mosquito vector, Ae. Aegyptus during phase II trials. Furthermore, formalin-inactivated vaccines are expensive and inefficient. These vaccines may also require multiple, repeated vaccinations. Therefore, a need exists for improved immunogenic agents against CHIKV.

In order to be effective in resource-limited nations that are endemic for alphavirus and to combat an epidemic, an ideal alphavirus vaccine should be capable of inducing a rapid and long-lived immunity after a single dose, have a low risk of reactogenicity and reversion to virulence, and be inexpensive. Immunogenic compositions against arboviral diseases should have a low risk of transmission from immunized persons via mosquitoes in the event that viremia occurs, especially those used in non-endemic regions. Although replication-defective immunogenic candidates have been described that emphasize safety, none has been shown to induce rapid or long-lived immunity after a single dose, and some may be expensive to produce.

Certain embodiments herein include live, attenuated recombinant alphaviruses incapable of replicating in mosquitoes and mosquito cells yet capable of replicating in mammalian cells. In accordance with these embodiments, these constructs can be generated using internal ribosome entry site (IRES) sequences other than EMCV IRES sequences. It is noted that not all IRES sequences are substitutable for EMCV IRES because several sequences failed to achieve desired phenotype sought. Live, attenuated alphaviruses of the present disclosure were achieved by carefully selecting specific IRES sequences and placing translation control of alphaviral replication under the control of specific IRES sequences of various exogenous species. In accordance with these embodiments, the exogenous IRES sequences exclude EMCV IRES but can include, and are not limited to, IRES sequences of Flavivirus, Picornavirus, Togavirus, Coronavirus, Rhabdovirus, Filovirus, Paramyxovirus, Orthomyxovirus, Bunyavirus, Arenavirus, Retrovirus, Hepadnavirus, Pestivirus, Calicivirus, Reovirus, Parvovirus, Papovavirus, Adenovirus, Herpes virus, and/or Poxvirus. See U.S. Pat. No. 8,426,188, disclosures of which are incorporated by reference in its entirety to the extent they are not inconsistent with the explicit teachings of this specification.

Picornaviruses (Picornaviridae) are non-enveloped positive strand RNA viruses with an icosahedral capsid, which cause many known human and/or animal diseases. Picornavirus contains some notable members, including rhinovirus, which infects humans more frequently than any other virus; poliovirus, which has paralyzed or killed millions over the years; and foot-and-mouth-disease virus, which led to the production of dedicated institutes throughout the world.

Picornaviruses contain internal ribosome entry site (IRES) elements that are cis-acting RNA regulatory sequence combined with a capacity to control cap-independent translation initiation in mRNAs when cap-dependent translation is compromised. The picornavirus IRES sequences are classified into three types based on their primary and secondary structures. Picornaviruses having Type I IRES sequences include, but are not limited to, enteroviruses: subtype, enterovirus 71 (EV71), human rhinovirus (HRV), human enterovirus A (HEV), coxsackievirus B (CVB) (human enterovirus B), poliovirus (PV) (human enterovirus C), bovine enterovirus (BEV). Picornaviruses having Type II IRES sequences include, but are not limited to, aphthoviruses: subtype, foot-and-mouth disease virus (FMDV), equine rhinitis A virus (ERAV), and bovine rhinitis B virus (BRBV); cardioviruses: subtype, Theilovirus (TMEV) and encephalomyocarditis virus (EMCV); erbovirus: subtype, Equine rhinitis B virus (ERBV); parechovirus: subtype, Human parechovirus (HPeV); kobuvirus: subtype, Aichi virus (AiV); avisivirus, cosavirus, and hunnivirus. These IRES types differ in host protein requirements as well as in the positions of the initiation codons with regard to the entry sites. The IRES of hepatitis A virus (HAV) is distinct from that of other picornaviruses and makes up a group (type III) on its own.

In some embodiments, Type I or Type II Picornavirus IRES sequences can be inserted into live, attenuated, recombinant alphaviruses. In other embodiments, live, attenuated, recombinant alphaviruses can include an IRES sequence from non-picornaviruses. These non-picornavirus IRES sequences can include, but are not limited to, IRES sequences from flavivirus: subtype, hapacivirus (HCV), pestivirus, dicistrovirus, and retrovirus. Some of these IRES sequences were used successfully to create alphavirus constructs of use for immunogenic compositions disclosed herein.

In certain embodiments, live, attenuated alphaviruses and alphavirus constructs can include, but are not limited to, alphaviruses such as chikungunya virus, o'nyong'nyong virus, Ross River virus, Eastern equine encephalitis Virus, Venezuelan equine encephalitis virus, Western equine encephalitis virus or other alphaviruses in the Coronaviridae and Togaviridae families. Other Semliki Forest virus complexes include, but are not limited to, Bebaru virus, Mayaro virus, Subtype: Una virus, O'Nyong Nyong virus: Subtype: Igbo-Ora virus, Ross River virus: Subtype: Bebaru virus; Subtype: Getah virus; Subtype: Sagiyama virus, Semliki Forest virus: Subtype: Me Tri virus or combinations thereof or combinations with other live, attenuated virus formulations.

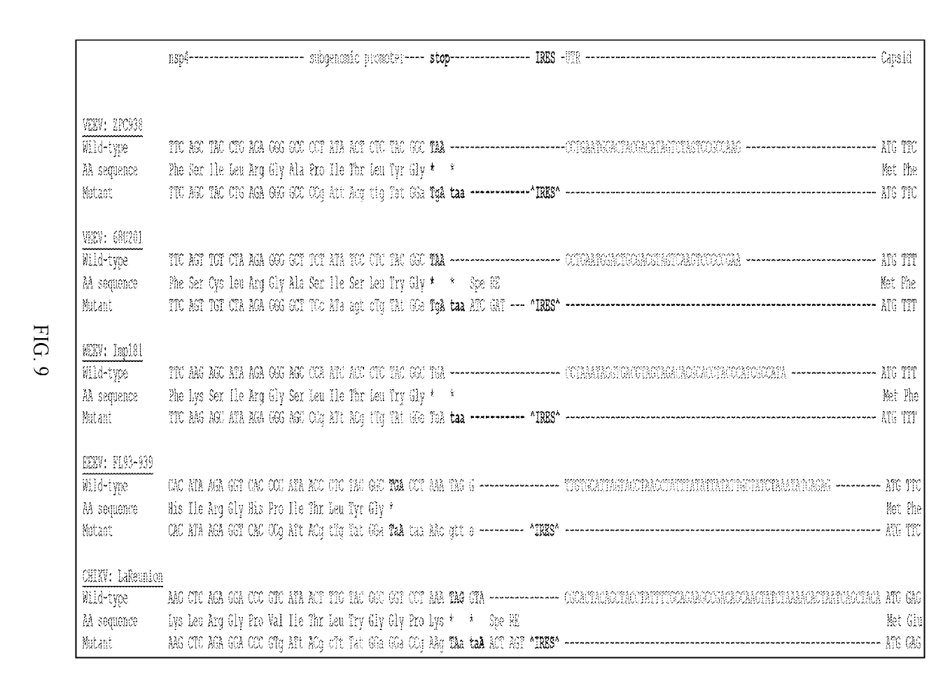

Some embodiments disclosed herein can include live, attenuated recombinant alphaviruses that are incapable of replicating in mosquito or other insect cells. In certain embodiments, a subgenomic promoter of live, attenuated recombinant alphaviruses disclosed herein can be inactivated by a deletion of the 5′ UTR of the subgenomic RNA between the end of nonstructural protein 4 (nsP4) coding sequence and initiating AUG of a subgenomic RNA coding sequence. In another embodiment, a subgenomic promoter of live, attenuated recombinant alphaviruses disclosed herein can be inactivated by clustered point mutations within the subgenomic promoter. In accordance with these embodiments, the alphavirus subgenomic promoter can include cluster point mutations of nucleic acids located in the 5′UTR of the subgenomic RNA. In other embodiments, an alphavirus subgenomic promoter can be inactivated using synonymous mutations while preserving wild-type amino acids of the non-structural proteins. In one example, the alphavirus, chikungunya virus, subgenomic promoter can be inactivated by clustered point mutations. Similar clustered point mutations can be used to inactivate subgenomic promoters of other alphaviruses. In accordance with these embodiments, clustered point mutations can include synonymous point mutations of two or more mutations at positions 7474, 7477, 7480, 7481, 7483, 7486, 7489, 7492, 7495, 7498, and/or 7501-7503 of at least one polynucleotide sequence encoding a polypeptide, the polynucleotide can include one or more of SEQ ID NO:1, SEQ ID NO:2, SEQ ID NO:3, SEQ ID NO:4, and SEQ ID NO:5. Clustered point mutations of use to inactivate the subgenomic promoter are at a sufficient number to reduce or prevent reversion, such as 2 or more, 4 or more, 6 or more of the above indicated mutations or up to all 12 mutations in the subgenomic promoter of the alphavirus target. Alphaviruses in certain embodiments disclosed herein are genetically similar (see for example, FIG. 9, an alignment of some alphaviruses).

Some embodiments disclosed herein include methods for manufacturing live attenuated, recombinant alphaviruses that are incapable of replicating in mosquitoes and mosquito cells yet capable of being expressed in mammalian cells. In accordance with these embodiments, these live attenuated, recombinant alphaviruses can be generated by introducing a mutation into an alphavirus genome, inactivating cap-dependent translation of a structural protein (or structural proteins) of the alphavirus, and cloning an IRES that selectively initiates translation in cells of vertebrate origin into the alphavirus genome upstream of the structural genes.

In other embodiments, live, attenuated recombinant alphaviruses disclosed herein can include: a) a capsid gene positioned downstream from envelope glycoprotein genes and upstream from the 3′ UTR of the alphavirus; b) at least one IRES introduced between the 3′ end of the envelope glycoprotein genes and the positioned capsid gene and wherein the genes are positioned 5′ to 3′ as enveloped glycoprotein genes, an inserted IRES, positioned capsid gene and 3′ UTR; and c) the envelope glycoprotein genes positioned upstream of the IRES are translated in an IRES-independent manner while the positioned capsid is translated in an IRES-dependent manner.

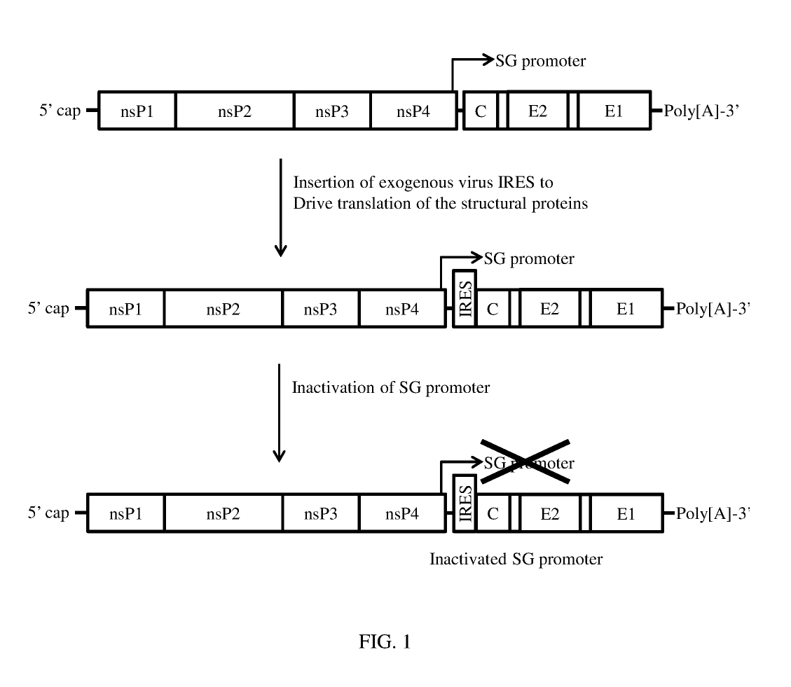

Referring to FIG. 1, where a genome of an alphavirus is illustrated, the alphavirus represented by the schematic diagram encodes 4 non-structural proteins (nsP1-4) and 3 major structural proteins (Capsid, E1, and E2 envelope glycoproteins). During replication, two distinct RNA's are produced: the genomic and subgenomic RNAs. The subgenomic RNA is transcribed late during infection from a subgenomic promoter (SG promoter), which can be found in the 3′ end of the nsP4 gene. Some embodiments include methods of generating live, attenuated, recombinant alphaviruses that are incapable of replicating in mosquito cells by inserting an IRES sequence of an exogenous virus or human/mammalian IRES (e.g., picornavirus and/or flavivirus) directly downstream from the SG promoter of an alphavirus cDNA clone (FIG. 1; middle figure). Other embodiments disclosed herein can also include live, attenuated, recombinant alphaviruses having an inactivated SG promoter. For example, the subgenomic promoter can be inactivated (e.g. one or more point mutations, FIG. 1 bottom). These live, attenuated, recombinant alphaviruses having an inactive SG promoter are stable in terms of its inability to revert to mosquito element-dependent translation. Because such reversion would require not only the IRES deletion, but also the restoration of a functioning subgenomic promoter making this a highly unlikely event.

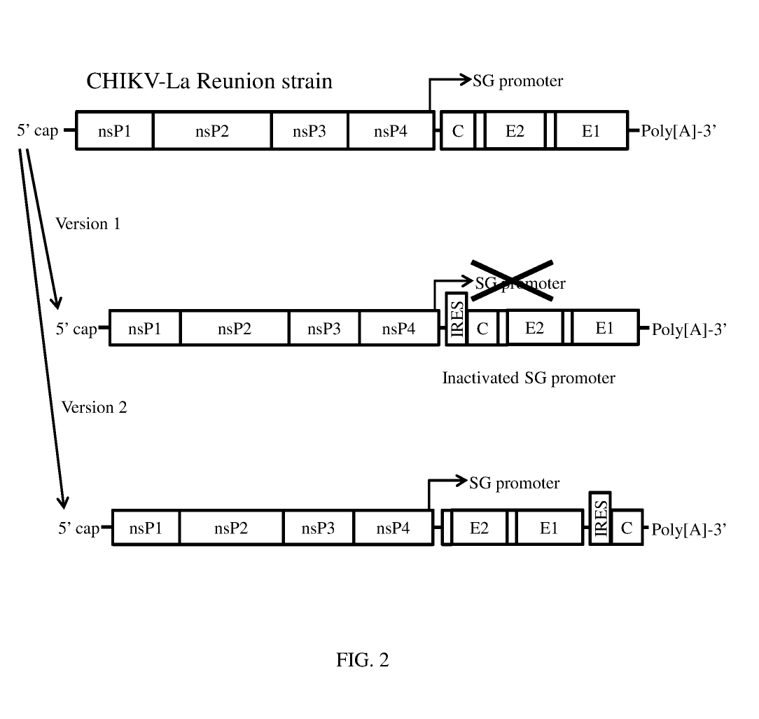

Referring now to FIG. 2, some embodiments disclosed herein can include methods of generating live, attenuated, recombinant alphaviruses by placing an IRES sequence downstream of the envelope glycoprotein genes, with the capsid gene at the 3′ end of the subgenomic region just upstream of the 3′ UTR. (FIG. 2; Version 2). These live, attenuated, recombinant alphaviruses produced by this strategy may retain an active subgenomic promoter. In these examples, a subgenomic message can be made, with the envelope protein genes translated in a cap-dependent manner and the capsid protein translated in an IRES-dependent manner. Other embodiments disclosed herein can include live, attenuated, recombinant alphaviruses having an inactive SG promoter. Based on one attenuation strategy described herein, live, attenuated chikungunya viruses were previously designed, that employed the encephalomyocarditis virus (EMCV) IRES to alter gene expression and attenuate the alphavirus. In one live, attenuated alphavirus, Chikungunya with an IRES insertion, clustered point mutations were used to inactivate the subgenomic promoter, and the IRES was inserted in the intergenic region upstream of the structural protein open reading frame (ORF). The second version CHIKV/IRESv2 retained a wild-type subgenomic promoter but positioned the capsid protein gene downstream of the envelope protein genes and behind the IRES. In some embodiments, live, attenuated, recombinant alphaviruses presented herein can be generated using these versions of live, attenuated viruses using the attenuated strategy as described. See for example U.S. Pat. No. 8,426,188, disclosures of which are incorporated by reference in its entirety to the extent they are not inconsistent with the explicit teachings of this specification. Upon consideration of these prior constructs using EMCV IRES sequences, it was surprisingly found that not all IRES sequences could be used in the constructs previously designed. In addition, not all picornavirus derived IRES sequences of which EMCV IRES is a member were suitable substitutes in the live, attenuated recombinant alphaviruses disclosed herein. It was discovered that only specific IRES sequences would function in the live, attenuated alphaviruses to attenuate the alphavirus while maintaining viability. Surprisingly, only some of the tested Type I and Type II picornavirus IRES sequences produced functional constructs and the Type III picornavirus IRES (HAV, hepatitis A) sequence did not work. In addition, a cellular FGF (e.g. mammalian) IRES sequence also failed to produce viable alphaviruses when inserted into constructs disclosed herein.

IRES sequences include extensive stem-loop/hairpin sequences of secondary structure created in the RNA. These structures are not shared across all IRES types, and are in fact quite different between the three types (Type I, II, and III). However, Type III differs more dramatically in secondary structure to Types I and II. This may have an impact on which translation factors are able to bind to the IRES structure and initiate polypeptide production from these promoters (See, for example, Martinez-Salas E. et al., Picornavirus IRES elements: RNA structure and host protein interactions, Virus Research, vol. 206, pp. 62-73, 2015, disclosures of which are incorporated by reference in its entirety to the extent they are not inconsistent with the explicit teachings of this specification.) In addition, it appears other structural differences within the three types of the picornavirus IRES sequences may contribute to the differing effects when generating alphavirus constructs of use in immunogenic compositions disclosed herein.

### Pharmaceutical Compositions

Embodiments herein provide for methods for making and using live, attenuated alphavirus constructs for administration to a subject in need in a biologically compatible form. By biologically compatible form suitable for administration in vivo can be a form of the active agent (e.g. live, attenuated alphavirus composition of certain embodiments) to be administered in which any toxic effects are outweighed by the therapeutic benefit of the active agent. Administration of a therapeutically active amount of the therapeutic compositions can be considered an amount effective, at dosages and for periods of time necessary to achieve a desired result. For example, a therapeutically active amount of a compound may vary according to factors such as the disease state, age, sex, and weight of the individual, and the ability formulations to elicit a desired response in the individual. Dosage regime may be adjusted to provide the optimum therapeutic response.

In some embodiments, compositions disclosed herein (e.g. pharmaceutical immunogenic composition of some embodiments) can be administered by any mode known to work for such a compositions, including, but not limited to, subcutaneous, intravenous, by oral administration, inhalation, transdermal application, intradermal application, intravaginal application, topical application, intranasal or rectal administration. In other embodiments, compositions disclosed herein can be administered subcutaneously administered. In yet another embodiment, compositions disclosed herein can be administered subcutaneously or intramuscularly or intradermally or by intranasal administration or by a combination as an initial dose and boost regimen.

An immunogenic composition can be administered to a subject in an appropriate carrier or diluent, co-administered with the composition. The term “pharmaceutically acceptable carrier” as used herein is intended to include diluents such as saline and aqueous buffer solutions. Live, attenuated alphavirus constructs disclosed herein can also be administered parenterally or intraperitoneally. Dispersions can also be prepared in appropriate buffers, such as HEPES buffer and other suitable agents. Under ordinary conditions of storage and use, these preparations may contain a preservative to prevent the growth of microorganisms.

Pharmaceutical compositions suitable for administration can be administered by any means known in the art. For example, sterile aqueous solutions (where water soluble) or dispersions and sterile powders or lyophilized/freeze-dried cakes for the extemporaneous preparation of sterile injectable solutions or dispersion may be used. In all cases, the composition can be sterile and can be fluid to the extent that easy syringability exists. It may further be preserved against the contaminating action of microorganisms such as bacteria and fungi. The pharmaceutically acceptable carrier can be a solvent, stabilizing composition or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Sterile injectable solutions can be prepared by incorporating active compound in an amount with an appropriate solvent or with one or a combination of ingredients enumerated above, as required, followed by sterilization.

Upon formulation, solutions can be administered in a manner compatible with the dosage formulation and in such amount as is therapeutically effective. A therapeutically effective amount is an amount of a biologically active compound that has a single or cumulative beneficial effect on the health or well-being of a patient. The formulations are easily administered in a variety of dosage forms, such as the type of injectable solutions described above. It is contemplated that slow release capsules, timed-release microparticles, and the like can also be employed for administering compositions herein. These particular aqueous solutions are especially suitable for intravenous, intramuscular, subcutaneous and intraperitoneal administration.

In order for live, attenuated viral vaccines to be effective, they must be capable of replicating after immunization. Any factors that inactivate the virus can cripple the live, attenuated alphavirus. Some of the commonly used vaccines are sensitive to temperature extremes; either excessive heat or accidental freezing can inactivate the vaccine. Maintaining this “cold chain” throughout distribution is particularly difficult in resource-limited nations that are endemic for alphavirus. In certain embodiments, live, attenuated, recombinant alphaviruses disclosed herein can be stored in as a liquid or aqueous formulation or frozen or lyophilized form to reduce or prevent deterioration or inactivation of these alphaviruses. In other embodiments, live, attenuated, recombinant alphaviruses disclosed herein can be formulated using various additives to maintain stability for extended periods of time at room temperatures (e.g. about 20° C. to about 25° C. or even as high as 40° C.) or refrigeration temperatures (e.g. about 0° to about 10° C.). Additives that reduce deterioration or inactivation of live, attenuated, recombinant alphaviruses can include, but are not limited to, HEPES (e.g. about 10.0 to about 200 mM HEPES) with carbohydrates and/or amino acids or PBS buffer or other suitable buffer. In certain embodiments, protein agents can be added to the formulations that can include, but not limited to, gelatin or other agents that have reduced allergic or immunogenic responses, essentially inert. Yet other embodiments herein are directed to reducing the need for lower temperatures (e.g. refrigerated or frozen storage) and increasing shelf life of aqueous and/or reconstituted live, attenuated, recombinant alphaviruses where the formulations of use to preserve the live, attenuated alphavirus can include HEPES buffer or other suitable buffer.

The active therapeutic agents may be formulated within a mixture can include live, attenuated alphaviruses measured by plaque forming units (PFUs) that induce an immune response (e.g. produce neutralizing antibodies) to a targeted alphavirus per dose. Single dose or multiple doses can also be administered on an appropriate schedule for a predetermined situation. In some embodiments, doses can be administered before, during and/or after exposure to a virus contemplated herein. In other embodiments, log PFU can vary depending on the subject administered the composition. Ranges of PFUs per dose can be between 2 Log₁₀ to 8 Log₁₀. In certain embodiments, a dose range can be between 3 Log₁₀ and 6 Log₁₀ when administered to a subject in order to induce an immune response to the alphavirus in the subject or other dose as found to be appropriate by a health professional.

In certain embodiments, live, attenuated alphavirus immunogenic compositions of use herein can be part of a pharmaceutical composition and can include either live, attenuated alphaviruses represented by polynucleotides, polypeptides or a mixture of both polynucleotides and polypeptides. In accordance with these embodiments, compositions disclosed herein can be used to treat a subject in need of such a treatment or for prevention of onset of a disorder or infection caused by exposure to an alphavirus.

### Kits

In some embodiments, live, attenuated recombinant alphaviruses, constructs and compositions disclosed herein can be part of a kit including, but not limited to at least one live, attenuated, recombinant alphavirus or construct disclosed herein and at least one container. In yet other embodiments, kit can further include, but are not limited to, one or more stabilizing agents, buffer, and/or delivery device. Kits can also include one or more additional agents suitable to be delivered with the compositions disclosed herein.

The kits may further include a suitably aliquoted composition of use in a subject in need thereof. In addition, compositions herein may be partially or wholly dehydrated or aqueous. Kits contemplated herein may be stored at room temperatures or at refrigerated temperatures as disclosed herein depending on the particular formulation.

The container means of the kits will generally include at least one vial, test tube, flask, bottle, syringe or other container means, into which a composition may be placed, and preferably, suitably aliquoted. Where an additional component is provided, the kit will also generally contain one or more additional containers into which this agent or component may be placed. Kits herein will also typically include a means for containing the agent, composition and any other reagent containers in close confinement for commercial sale. Such containers may include injection or blow-molded plastic containers into which the desired vials are retained.

### EXAMPLES

The following examples are included to demonstrate certain embodiments presented herein. It should be appreciated by those of skill in the art that the techniques disclosed in the Examples which follow represent techniques discovered to function well in the practices disclosed herein, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope herein.

### Example 1

### Production of Plasmid and Sequencing

In one exemplary method, CHIKV cDNA clone containing the EMCV IRES with the subgenomic promoter ablated using inactivating mutations (CHIKV/IRES=the EMCV IRES-containing positive control virus) was produced using standard recombinant DNA techniques in which the infectious clone of La Reunion strain (LR) described previously was used as a template and a positive control for other constructs disclosed below. Inactivation of the subgenomic promoter was performed using site-specific mutagenesis. An intermediate construct encoding the 3′ end of the nsP4 gene through the subgenomic promoter was produced using PCR with high fidelity Phusion DNA polymerase from Finnzymes (Espoo, Finland). The resultant amplicon was cloned into a shuttle vector, prS2, and was sequenced using the BigDye kit (e.g. Applied Biosystems, Foster City, Calif.). The 5′ end of capsid gene from the LR strain was amplified using PCR with an overhang complementary to the IRES sequence. The IRES-containing and capsid fragments were then joined using fusion PCR, and this fragment was cloned back into the shuttle vector and resequenced. The IRES/Capsid fragment and the mutated subgenomic fragment were finally ligated together through the Spel site introduced into both fragments. The completed insert was then cloned into the LR backbone and this final construct was completely sequenced.

Further, the CHIKV cDNA clones containing Picornavirus Type I IRES sequences with the subgenomic promoter inactivated using inactivating mutations (e.g., EV71 and HRV), the CHIKV cDNA clones containing Picornavirus Type II IRES sequences with the subgenomic promoter inactivated using inactivating mutations (e.g., FMDV and TMEV), the CHIKV cDNA clones containing Picornavirus Type III IRES sequences with an inactivated subgenomic promoter using inactivating mutations (e.g., HAV), the CHIKV cDNA clones containing non-picornavirus IRES sequences with the subgenomic promoter ablated using inactivating mutations (e.g., HCV), and the CHIKV cDNA clones containing non-viral, mammalian IRES with an inactivated subgenomic promoter using inactivating mutations (e.g., FGF1), were produced using standard recombinant DNA techniques described supra.

### Example 2

### RNA Transcriptions, Transfections, and Virus Production

Large-scale plasmid purification was done using anion exchange columns. The purified DNA was then linearized using Notl restriction endonuclease (e.g. New England BioLabs, Ipswich, Mass.), and a small sample was analyzed on a 1.2% agarose gel to verify linearization. The remaining DNA was transcribed using an Ambion SP6 In vitro transcription kit. The RNA was quantified and used to electroporate Vero cells using a BTX ECM 830 electroporator. Briefly, two T-150 flasks containing 90% confluent Vero cells were trypsinized and washed 3 times in RNAse-free DPBS. The cells were resuspended in 700 μl of DPBS and 10 μg of RNA was added. The solution was placed in a 4 mm cuvette and was pulsed 2 times at 250 v for 10 msec at 1 sec intervals. The cells were then left at room temperature for 10 minutes before being plated in T-75 flasks. The viruses were harvested at 48 hours post-electroporation or until obvious CPE was observed and centrifuged at 771×g. Supernatant was collected and titered by TCID₅₀ assay on Vero cells.

### Cell Cultures

In one example, Vero African green monkey kidney cells were obtained (e.g. from the American Type Cell Culture (Bethesda, Md.)). The cells were maintained at 37° C. in Dulbecco's Modified Eagle Medium (DMEM) supplemented with 10% fetal bovine serum (FBS), penicillin and streptomycin. C6/36 Ae. albopictus cells were also maintained in DMEM containing 10% FBS at 28° C.

### Cell Culture Growth Curves

Replication kinetics was measured in T-25 flasks with duplicates for each virus tested. The flasks were seeded to a confluency of 95% using Vero or C6/36 cells. Media was removed and they were infected at an MOI of 0.001 for one hour. Then 5 ml of DMEM containing 5% FBS was added. A 0 time point was immediately removed (300 μl). At each of the remaining time points on days 1-6 300 μl was removed. The samples were titered by TCID₅₀ assay.

### Example 3

### Virus and Antibody Titers

In other exemplary experiments, depending on containment requirements and sensitivity needs, alphavirus stocks and experimental samples were titered by plaque assay or TCID₅₀ or were estimated using quantitative real-time PCR with dilutions of virus to generate standard curves from which (plaque forming unit) PFU titers could be extrapolated. In this particular assay, primers were used, for example, (5′-GAYCCCGACTCAACCATCCT-3′-SEQ ID NO:15) and (5′-CATMGGGCARACGCACTGGTA-3′—SEQ ID NO:16) and the probe (5′-AGYGCGCCAGCAAGGAGGAKGATGT-3′—SEQ ID NO:17) containing an exemplary dye FAM. Ab titers were measured using plaque reduction neutralization tests with 50% reduction endpoints.

### Example 4

### Virus Rescue

In certain exemplary methods, recombinant alphavirus constructs with multiple mutations in the subgenomic promoter were generated in cDNA from using standard recombinant DNA techniques using the IRES-based attenuation strategy described supra. All resultant viruses (e.g. IRES from FMDV, HCV, EV71, TMEV, and HRV), rescued by electroporation of in vitro-transcribed RNA into Vero cells, contained non-functional subgenomic promoters. FGF1 and HAV IRES constructs did not exhibit any CPE. Further, constructs having an IRES sequence of FGF1 and HAV collected in Vero cells but did not show viral activity.

### Example 5

### Growth Kinetics on Vero Cells

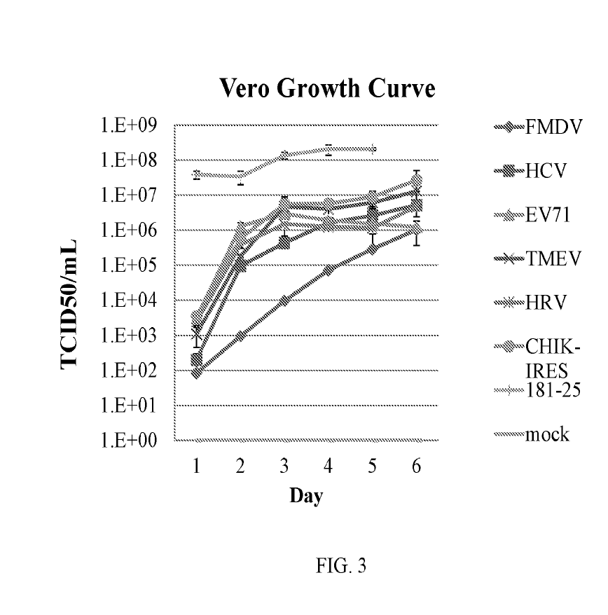

To assess viability in Vero Cells, viruses derived from the electroporation were compared after CHIKV (chikungunya virus) infection. Referring now to FIG. 3, CHIK-IRES and other live, attenuated alphaviruses were studied for viability in Vero cells and attenuation in growth relative to the investigational vaccine chikungunya 181-25 strain. Similarly, recombinant alphavirus constructs containing IRES sequences of FMDV, EV71, HCV, TMEV, and HRV were found to be viable in Vero cells and exhibited an attenuation level that is similar to CHIK-IRES in growth relative to classically attenuated (after passage in cell culture) 181-25 strain. HCV and FMDV showed attenuation in growth relative to CHIK-IRES. However, FGF1 and HAV IRES constructs did not generate viable virus. Growth kinetics on C6/36 Mosquito Cells

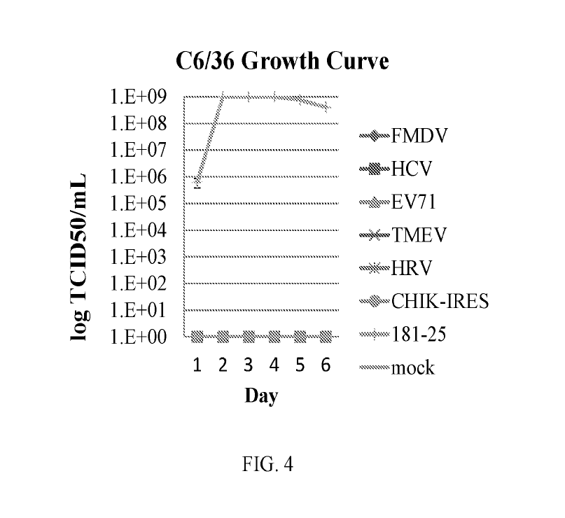

To assess viability in mosquito cells, viruses derived from the electroporation were compared after infection of C6/36 mosquito cells. Referring now to FIG. 4, the 181-25 strain was the only strain viable in C6/36 mosquito cells. All recombinant alphavirus constructs tested herein were not viable and did not exhibit any growth in C6/36 mosquito cells.

### Example 6

### Plaque Assay

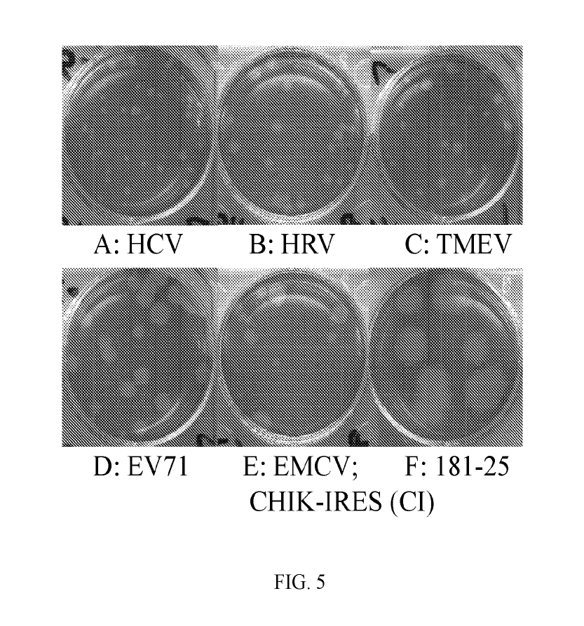

The chikungunya 181-25 strain and CHIK-IRES (EMCV IRES, positive control) generated plaques on Vero cells. Referring now to FIG. 5, some recombinant alphavirus constructs (e.g., IRES from EV71, HCV, TMEV, and HRV) generated plaques on Vero cells, but exhibited difference sizes of plaques. For example, constructs having an EV71 IRES sequence insert generated consistently larger plaques than control CHIK-IRES; whereas, other recombinant alphavirus constructs such as construct with IRES sequences from HCV, HRV, and TMEV generated plagues that smaller than EV71 IRES sequence insert construct, CHIK-IRES (EMCV, positive control construct), and the 181-25 strain. FMDV failed to produce plaques. Plaque size is an indicator of viral fitness, and each construct had unique plaque phenotypes—in further support that not all IRES insertions perform equally in this method of attenuation producing.

### Example 7

### Neutralization Titers

Neutralizing rabbit polyclonal antibodies raised against the chikungunya virus, 181-25, are publically available. This antibody was tested in a TCID₅₀ neutralization assay for the ability of each of these IRES constructs to be neutralized to this reagent. Each live, attenuated alphavirus strain was tested for its ability to neutralize the neutralizing polyclonal antibody (nPab). It is understood that this antibody binds to epitopes on the virus that have the ability to induce an immunogenic and neutralizing antibody response in vivo. A constant amount of virus (1×10⁴ TCID₅₀) was added to a 2-fold dilution series of antibodies. The dilution at which 50% of the input virus was neutralized is compared between each live, attenuated alphavirus strain.

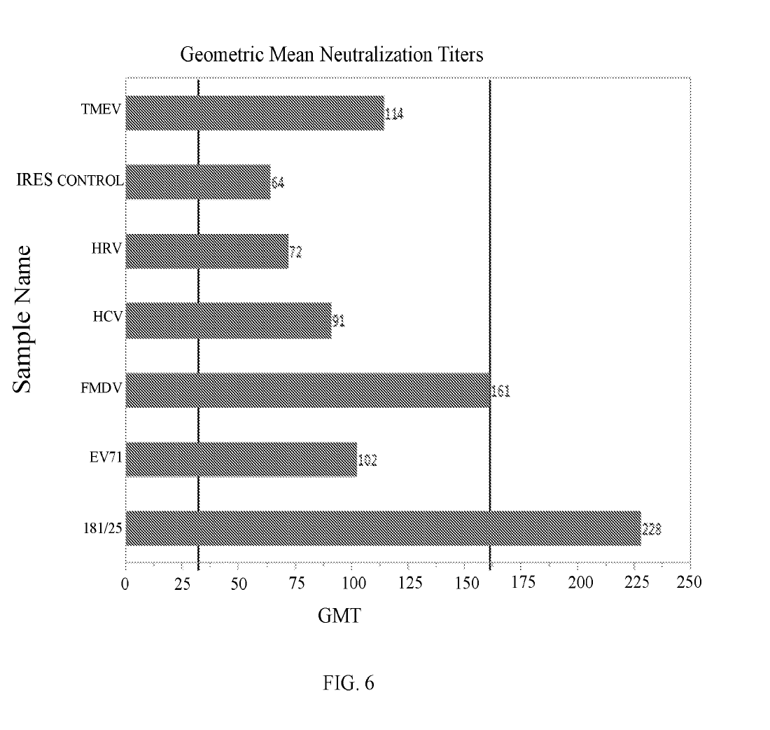

Referring now to FIG. 6, as illustrated in this histogram plot, a control anti-chikungunya rabbit serum pool was serially diluted from 1:4 through 1:8192. The five recombinant alphavirus constructs, the chikungunya-EMCV IRES (IRES CONTROL, CHIK/IRES), and a 181-25 army chikungunya vaccine strain were diluted to equal virus titers of 2000 TCID₅₀/mL. Equivalents volumes of each antibody dilution and each virus sample were combined and incubated at 37° C. for 1.5 hours. 100 ul of each virus-antibody sample was plated per well of a 96-well Vero cell plate. After 5 days, the highest dilution of antibody where the virus was fully neutralized, as indicated by no CPE present in the cell layer, was recorded. The values in the figure are the geometric means of three replicates on two different occasions. The EMCV IRES control resulted in GMT values from 32-161, as indicated by the vertical lines in the graph.

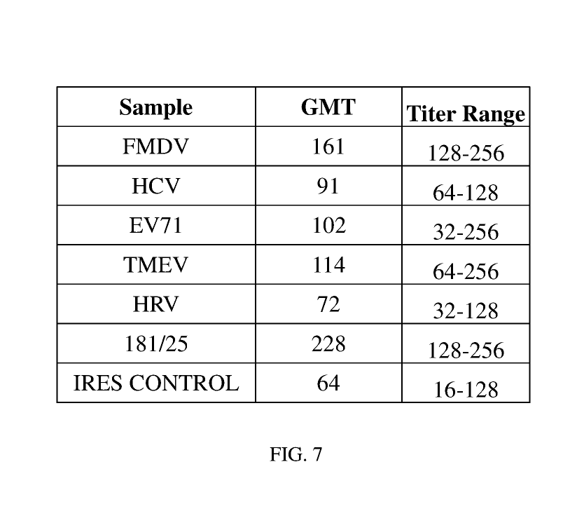

Referring now to FIG. 7, a control anti-chikungunya rabbit serum pool was serially diluted from 1:4 through 1:8192. The tested IRES alphavirus constructs, the chikungunya EMCV-IRES (IRES CONTROL), and a classically attenuated live 181/25 army chikungunya vaccine strain were diluted to equal virus titers of 2000 TCID₅₀/mL. Equivalents volumes of each antibody dilution and each virus sample were combined and incubated at 37° C. for 1.5 hours. 100 ul of each virus-antibody sample was plated per well of a 96-well Vero cell plate. After 5 days, the highest dilution of antibody where the virus was fully neutralized, as indicated by no CPE present in the cell layer, was recorded. The values in the figure are the geometric means of three replicates on two different experiments. Each of the viable alphavirus constructs were able to be neutralized by the neutralizing antibody. The GMT were not equivalent across vaccine genotypes, but the titer ranges were within 1 or 2 dilutions—indicating robust neutralization.

### Example 8

### Animal Studies

Mice can be used in a preliminary animal study. For example, A129 mice can be obtained and used for mouse studies similar to previous studies of the control construct, EMCV-IRES alphavirus constructs. Animals 3 to 10 weeks of age can be infected with about 1×10⁴ PFU of a selected construct or other suitable dose intradermally in the left rear footpad. Footpad measurements can be taken 48 hours post vaccination with a caliper as the vertical height of the hind feet at the balls. The animals can be maintained for 15 to 45 days (or about 38 days) and bled on days 21 and 35. These animals can then be challenged with about 100 PFU or other suitable challenge of alphavirus (e.g. CHIKV, naturally-occurring and monitored for symptoms of infection, morbidity and mortality. Reactions to these challenges can be observed for reduced infection and protection against infection by CHIKV or other target alphavirus.

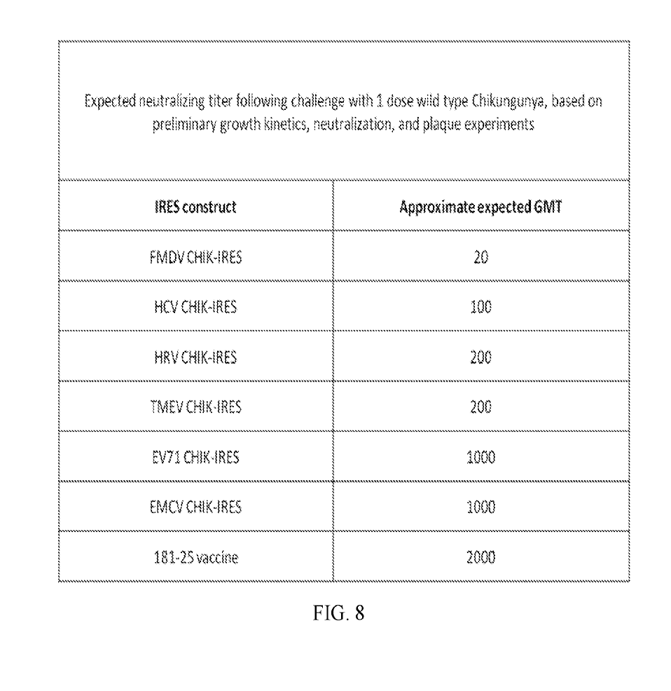

Based on the preliminary data, mice immunized with any of the five constructs (See FIG. 7 or 8) would respond to CHIK wild-type challenge with protection against viral infection and subsequent viral infectious conditions. Based on these combined data, mice will produce a neutralizing titer trend illustrated in table found in FIG. 8, measured in GMT. EV71 IRES alphavirus constructs will likely perform as well if not better than the control EMCV CHIK-IRES based in part on its ability to generate consistently larger plaques than the other constructs tested, including EMCV CHIK-IRES. All test constructs illustrated in FIG. 8 will likely produce neutralizing titer in a subject receiving such a construct suitable to reduce infection of a target alphavirus in the subject when exposed to the target alphavirus.

### Phase I Study Design

In another example, a phase I, open-label, placebo-controlled dose-escalation first-in-human single centered trial with a genetically modified live-attenuated chikungunya vaccine candidate is described where 44 healthy male and female adults aged 18 to 49 years are assessed in the study. Informed consent procedure, eligibility assessment for trial participation and pre-vaccination blood draw (for baseline routine laboratory parameters, to exclude HIV/chronic hepatitis and for baseline chikungunya antibody titers) will be performed at Day 0 occurring up to 28 days prior to vaccination with a single dose of the CHIKV vaccine candidate at Day 1. Safety, tolerability and immunogenicity will be assessed 7, 14, 28, 182 and 364 days post vaccination. Because of the preliminary studies discussed above on each of the test constructs illustrated in FIG. 8 and FIG. 9, it is likely that all constructs will produce neutralizing antibodies to CHIK in the subject post administration of the construct.

### Example 9

### Mosquito Infections

An Ae. albopictus colony from mosquitoes can be collected and used for these experiments. This species can be selected because these insects are highly susceptible to the LR CHIKV strain (and other alphaviruses). Adult female mosquitoes collected 3-4 days post-eclosion can be anesthetized using a chill table (Bioquip, Rancho Dominguez, Calif.) and can be injected intrathoracically with ca. 1.0 μL of a 10⁴ Vero PFU/ml virus stock. The mosquitoes can be incubated for 7 days at 27° C. with 10% sucrose provided ad libitum. The mosquitoes can be frozen and triturated in a medium, for example, MEM containing 2% FBS and fungicide. Following centrifugation for 10 minutes at 10,000×G, the supernatant can be plated on Vero cells using 96 well plates. The cells can be infected for 1 hour at 37° C. and then covered with 2% FBS containing MEM and allowed to incubate for 48 hr. to measure CPE. Then the cells will be assessed for presence of a target construct to assess infectivity of insect cells by the construct. Based on the preliminary evidence presented above, it is likely that none of the test constructs illustrated in FIG. 8 and FIG. 9 will produce significant levels of live, attenuated alphavirus in the insect cells but as demonstrated the constructs are capable of growth in mammalian cells.

All of the COMPOSITIONS and METHODS disclosed and claimed herein can be made and executed without undue experimentation in light of the present disclosure. While the compositions and methods have been described in terms of preferred embodiments, it is apparent to those of skill in the art that variations maybe applied to the COMPOSITIONS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. A live, attenuated, recombinant alphavirus comprising; an alphavirus nucleic acid, having (i) an inactivated subgenomic promoter, and (ii) an insertion of an internal ribosomal entry site (IRES) between one end of nonstructural protein 4 (nsP4) coding sequence and initiating AUG of a subgenomic RNA coding sequence of the alphavirus; wherein the at least one virus IRES comprises Type I or Type II Picornavirus IRES or other IRES sequence capable of producing a viable live, attenuated alphavirus.

2. The attenuated alphavirus according to claim 1, wherein the IRES sequence is selected from the group consisting of: enterovirus IRES, enterovirus 71 IRES (EV71 IRES), rhinovirus IRES, human rhinovirus IRES (HRV IRES), aphthovirus IRES, aphthovirus: subtype: foot-and-mouth disease virus (FMDV IRES), Theilovirus IRES (TMEV IRES), and flavivirus IRES, flaviviruses subtype: Hepacivirus (HCV IRES); and wherein the alphavirus is attenuated.

3. The attenuated alphavirus according to claim 1 or 2, wherein the subgenomic promoter is inactivated by a deletion of the 5′ UTR of the subgenomic RNA between the end of nonstructural protein 4 (nsP4) coding sequence and initiating AUG of a subgenomic RNA coding sequence.

4. The attenuated alphavirus according to claim 1 or 2, wherein the subgenomic promoter is inactivated by clustered point mutations located in the carboxy terminus of nonstructural protein 4 in the alphavirus.

5. The attenuated alphavirus according to any of claims 1-4, wherein the live alphavirus comprise one or more of chikungunya virus (CHIK), o'nyong'nyong virus, Ross River virus, Eastern Equine Encephalitis Virus, Venezuelan Equine Encephalitis Virus, Western Equine Encephalitis virus or other alphaviruses in the Coronaviridae and Togaviridae families, Semliki Forest virus complexes comprising Bebaru virus, Mayaro virus, Subtype: Una virus, O'Nyong Nyong virus: Subtype: Igbo-Ora virus, Ross River virus: Subtype: Bebaru virus; Subtype: Getah virus; Subtype: Sagiyama virus, Semliki Forest virus: Subtype: Me Tri virus and combinations thereof.

6. The attenuated alphavirus according to claim 4, wherein clustered point mutations are synonymous point mutations that include two or more point mutations at position 7474, 7477, 7480, 7481, 7483, 7486, 7489, 7492, 7495, 7498, 7501, 7502, and 7503 of at least one polynucleotide sequence selected from the group consisting of SEQ ID NO:1, SEQ ID NO:2, SEQ ID NO:3, SEQ ID NO:4, and SEQ ID NO:5.

7. The attenuated alphavirus according to any one of claim 3, 4 or 6, wherein the mutation of the subgenomic promoter does not modify the amino acid sequence of the carboxy terminus of nonstructural protein 4.

8. The attenuated alphavirus according to any one of claims 1-7, further comprising adaptive mutations in any one of the non-structural proteins of the alphavirus, wherein the adaptive mutations can be selected for increase virus replication, release and virus titers.

9. The attenuated alphavirus according to any one of claims 1-8, wherein the IRES comprises an enterovirus 71 IRES (EV71 IRES).

10. The attenuated alphavirus according to any one of claims 1-8, wherein the IRES comprises a human rhinovirus IRES (HRV IRES).

11. The attenuated alphavirus according to any one of claims 1-8, wherein the IRES comprises a foot-and-mouth disease virus IRES (FMDV IRES).

12. The attenuated alphavirus according to any one of claims 1-8, wherein the IRES comprises a Theilovirus IRES (TMEV IRES).

13. The attenuated alphavirus according to any one of claims 1-8, wherein the IRES comprises a hepacivirus IRES (HCV IRES).

14. The attenuated alphavirus according to any one of claims 1-13, wherein the alphavirus is chikungunya virus.

15. The attenuated alphavirus according to any one of claims 1-13, wherein the alphavirus is Eastern equine encephalitis virus.

16. The attenuated alphavirus according to any one of claims 1-13, wherein the alphavirus is Venezuelan equine encephalitis virus.

17. The attenuated alphavirus according to any one of claims 1-13, wherein the alphavirus is Western equine encephalitis virus.

18. The attenuated alphavirus according to any one of claims 1-17, wherein the alphavirus is incapable of replicating in mosquitoes and mosquito cells.

19. A vector comprising a nucleotide sequence encoding the live, attenuated alphavirus according to any of claims 1-18.

20. A host cell comprising and expressing the vector of claim 19.

21. A pharmaceutical composition comprising: the attenuated recombinant alphavirus according to any of claims 1-18 and a pharmaceutically acceptable carrier.

22. The use of the pharmaceutical composition according to claim 21, for inducing an immune response in a subject in order to reduce the onset of an infections from the alphavirus from exposure to the alphavirus.

23. The use according to claim 22, wherein the subject is a human, livestock, or other domesticated animal or pet.

24. A method of manufacturing a live, attenuated recombinant alphavirus according to any of claims 1-18 that is incapable of infecting mosquitoes, comprising: cloning an internal ribosomal entry site (IRES) selected from the group consisting of: enterovirus IRES, rhinovirus IRES, aphthovirus IRES, a aphthovirus: IRES, and flavivirus IRES; between one end of nonstructural protein 4 (nsP4) coding sequence and initiating AUG of a subgenomic RNA coding sequence of the alphavirus.

25. The method according to claim 24, wherein the alphavirus is further attenuated by inactivating the subgenomic promoter.

26. The method according to claim 24 or 25, wherein the IRES comprises a EV71 IRES.

27. The method according to claim 24 or 25, wherein the IRES comprises a HRV IRES.

28. The method according to claim 24 or 25, wherein the IRES comprises a FMDV IRES.

29. The method according to claim 24 or 25, wherein the IRES comprises a TMEV IRES.

30. The method according to claim 24 or 25, wherein the IRES comprises a HCV IRES.

31. The method according to claim 24 or 25, wherein the live, attenuated recombinant alphavirus comprises one or more of chikungunya virus (CHIK), o'nyong'nyong virus, Ross River virus, Eastern equine encephalitis virus, Venezuelan equine encephalitis virus, Western equine encephalitis virus or other alphaviruses in the Coronaviridae and Togaviridae families, Semliki Forest virus complexes comprising Bebaru virus, Mayaro virus, Subtype: Una virus, O'Nyong Nyong virus: Subtype: Igbo-Ora virus, Ross River virus: Subtype: Bebaru virus; Subtype: Getah virus; Subtype: Sagiyama virus, Semliki Forest virus: Subtype: Me Tri virus and combinations thereof.

32. A method for inducing an immune response in a subject to an alphavirus, comprising administering to the subject an attenuated recombinant alphavirus composition according to claim 21.

33. The method according to claim 32, wherein the subject is a human, livestock, a pet or other animal.

34. The method according to claim 32 or 33, wherein the live, attenuated recombinant alphavirus comprises one or more of chikungunya virus (CHIK), o'nyong'nyong virus, Ross River virus, Eastern equine encephalitis virus, Venezuelan equine encephalitis virus, Western equine encephalitis virus or other alphaviruses in the Coronaviridae and Togaviridae families, Semliki Forest virus complexes comprising Bebaru virus, Mayaro virus, Subtype: Una virus, O'Nyong Nyong virus: Subtype: Igbo-Ora virus, Ross River virus: Subtype: Bebaru virus; Subtype: Getah virus; Subtype: Sagiyama virus, Semliki Forest virus: Subtype: Me Tri virus and combinations thereof.

35. A kit, comprising:
at least one live, attenuated recombinant alphavirus of any one of claims 1-21; and
at least one container.

36. The kit according to claim 35, further comprising a composition comprising one or more stabilizing agents, wherein the composition decreases inactivation of the live, attenuated recombinant alphavirus.

37. A live, attenuated recombinant alphavirus comprising;
a) a capsid gene positioned downstream from envelope glycoprotein genes and upstream from the 3′ UTR of the alphavirus;
b) at least one IRES introduced between the 3′ end of the envelope glycoprotein genes and the positioned capsid gene and wherein the genes are positioned 5′ to 3′ as enveloped glycoprotein genes, inserted IRES, positioned capsid gene and 3′ UTR; and
c) the envelope glycoprotein genes positioned upstream of the IRES are translated in an IRES-independent manner while the positioned capsid is translated in an IRES-dependent manner;
wherein the at least one virus IRES is Type I or Type II Picornavirus IRES or other IRES sequence capable of producing a viable live attenuated alphavirus.

38. The attenuated alphavirus according to claim 1, wherein the IRES sequence is selected from the group consisting of: enterovirus IRES, rhinovirus IRES, aphthovirus IRES, Theilovirus IRES, and flavivirus IRES; and wherein the alphavirus is attenuated.

39. The attenuated alphavirus according to any one of claim 37 or 38, wherein the IRES comprises an enterovirus 71 IRES (EV71 IRES).

40. The attenuated alphavirus according to any one of claim 37 or 38, wherein the IRES comprises a human rhinovirus IRES (HRV IRES).

41. The attenuated alphavirus according to any one of claim 37 or 38, wherein the IRES comprises a foot-and-mouth disease virus IRES (FMDV IRES).

42. The attenuated alphavirus according to any one of claim 37 or 38, wherein the IRES comprises a Theilovirus IRES (TMEV IRES).

43. The attenuated alphavirus according to any one of claim 37 or 38, wherein the IRES comprises a hepacivirus IRES (HCV IRES).

44. The attenuated alphavirus according to any of claims 37 to 43, wherein the live, attenuated recombinant alphavirus comprises one or more of chikungunya virus (CHIK), o'nyong'nyong virus, Ross River virus, Eastern equine encephalitis virus, Venezuelan equine encephalitis virus, Western equine encephalitis virus or other alphaviruses in the Coronaviridae and Togaviridae families, Semliki Forest virus complexes comprising Bebaru virus, Mayaro virus, Subtype: Una virus, O'Nyong Nyong virus: Subtype: Igbo-Ora virus, Ross River virus: Subtype: Bebaru virus; Subtype: Getah virus; Subtype: Sagiyama virus, Semliki Forest virus: Subtype: Me Tri virus and combinations thereof.

45. The attenuated alphavirus according to any one of claims 37 to 43, wherein the live, attenuated alphavirus is chikungunya virus (CHIKV).


SEQUENCE LISTING (35 sequences)

SEQ ID NO: 1 - DNA, 12500 bp, Artificial Sequence
  CHIK-EV71 IRES
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgatgtgcaagactaccgacacggttgacggcgaaagaatgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcaacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgattgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgacctagtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtggcggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgattgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttaagactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtaaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtgattacgctttatggaggaccgaagtaataaactagtttaaaacagcctgtgggttgcacccacccacagggcccactgggcgctagcactctggtactgaggtacctttgtgcgcctgtttttactccccttcccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagagcctattgagctagctggtagtcctccggcccctgaatgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaattgtttacctatttattggttttgtaccattatcactgaagtctgtgatcactctcaaattcattttgaccctcaacacaatcaaacatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgcgcgcggtaccacaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtaaaggggaccatcgataacgcggacctggccaaactggcctttaagcggtcatctaagtatgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaaattaataaaaatcaaatgaataccataattggcaaacggaagagatgtaggtacttaagcttcctaaaagcagccgaactcactttgagaagtaggcatagcataccgaactcttccacgattctccgaacccacagggacgtaggagatgttattttgtttttaatatttc

SEQ ID NO: 2 - DNA, 12355 bp, Artificial Sequence
  CHIKV-HRV2 IRES
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgatgtgcaagactaccgacacggttgacggcgaaagaatgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcaacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgattgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgacctagtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtggcggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgattgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttaagactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtaaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtgattacgctttatggaggaccgaagtaataaactagtatccaggttgttcccacctggatttcccacagggagtggtactctgttattacggtaactttgtacgccagttttatctcccttcccccatgtaacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttttcctttatatttgcttatggtgacaatatatacaatatatatattggcaccatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgcgcgcggtaccacaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtaaaggggaccatcgataacgcggacctggccaaactggcctttaagcggtcatctaagtatgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaaattaataaaaatcaaatgaataccataattggcaaacggaagagatgtaggtacttaagcttcctaaaagcagccgaactcactttgagaagtaggcatagcataccgaactcttccacgattctccgaacccacagggacgtaggagatgttattttgtttttaatatttc

SEQ ID NO: 3 - DNA, 12216 bp, Artificial Sequence
  CHIKV-FMDV IRES
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgatgtgcaagactaccgacacggttgacggcgaaagaatgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcaacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgattgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgacctagtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtggcggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgattgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttaagactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtaaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtgattacgctttatggaggaccgaagtaataaactagtagcaggtttccccaactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccctatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgcgcgcggtaccacaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtaaaggggaccatcgataacgcggacctggccaaactggcctttaagcggtcatctaagtatgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaaattaataaaaatcaaatgaataccataattggcaaacggaagagatgtaggtacttaagcttcctaaaagcagccgaactcactttgagaagtaggcatagcataccgaactcttccacgattctccgaacccacagggacgtaggagatgttattttgtttttaatatttc

SEQ ID NO: 4 - DNA, 12310 bp, Artificial Sequence
  CHIKV-TMEV IRES
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgatgtgcaagactaccgacacggttgacggcgaaagaatgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcaacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgattgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgacctagtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtggcggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgattgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttaagactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtaaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtgattacgctttatggaggaccgaagtaataaactagtttggactccggtcccccggtctaagccgctcggaatatgacagggttattttcacctcttcttttctactccacagtgttctatactgtggaagggtatgtgttgccccttccttcttggagaacgtgcgcggcggtctttccgtctctcgacaagcgcgcgtgcaacatacagagtaacgcgaagaaagcagttctcggtctagctctagtgcccacaagaaaacagctgtagcgaccacacaaaggcagcggaacccccctcctggtaacaggagcctctgcggccaaaagccacgtggataagatccacctttgtgtgcggtgcaaccccagcaccctggtttcttggtgacactctagtgaacccctgaatggcaatctcaagcgcctctgtagggaagccaagaatgtccaggaggtaccccttcctctcggaagggatctgacctggagacacatcacacgtgctttacacctgtgcttgtgtttaaaaattgttacagcttccccgaaccaagtggtcttggttttcactttttatcacactgtcaatatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgcgcgcggtaccacaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtaaaggggaccatcgataacgcggacctggccaaactggcctttaagcggtcatctaagtatgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaaattaataaaaatcaaatgaataccataattggcaaacggaagagatgtaggtacttaagcttcctaaaagcagccgaactcactttgagaagtaggcatagcataccgaactcttccacgattctccgaacccacagggacgtaggagatgttattttgtttttaatatttc

SEQ ID NO: 5 - DNA, 12096 bp, Artificial Sequence
  CHIKV-HCV IRES
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgatgtgcaagactaccgacacggttgacggcgaaagaatgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcaacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgattgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgacctagtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtggcggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgattgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttaagactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtaaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtgattacgctttatggaggaccgaagtaataaactagtgccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgcgcgcggtaccacaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtaaaggggaccatcgataacgcggacctggccaaactggcctttaagcggtcatctaagtatgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaaattaataaaaatcaaatgaataccataattggcaaacggaagagatgtaggtacttaagcttcctaaaagcagccgaactcactttgagaagtaggcatagcataccgaactcttccacgattctccgaacccacagggacgtaggagatgttattttgtttttaatatttc

SEQ ID NO: 6 - DNA, 748 bp, Enterovirus 71
ttaaaacagcctgtgggttgcacccacccacagggcccactgggcgctagcactctggtactgaggtacctttgtgcgcctgtttttactccccttcccccgaagtaacttagaagctgtaaatcaacgatcaatagcaggtgtggcacaccagtcataccttgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagagcctattgagctagctggtagtcctccggcccctgaatgcggctaatcctaactgcggagcacatgctcacaaaccagtgggtggtgtgtcgtaacgggcaactctgcagcggaaccgactactttgggtgtccgtgtttccttttattcctatattggctgcttatggtgacaatcaaaaagttgttaccatatagctattggattggccatccggtgtgcaacagggcaattgtttacctatttattggttttgtaccattatcactgaagtctgtgatcactctcaaattcattttgaccctcaacacaatcaaacatg

SEQ ID NO: 7 - DNA, 604 bp, Human rhinovirus
atccaggttgttcccacctggatttcccacagggagtggtactctgttattacggtaactttgtacgccagttttatctcccttcccccatgtaacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttttcctttatatttgcttatggtgacaatatatacaatatatatattggcaccatgg

SEQ ID NO: 8 - DNA, 461 bp, Foot-and-mouth disease virus
agcaggtttccccaactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccct

SEQ ID NO: 9 - DNA, 960 bp, Theiler′s encephalomyelitis virus
ggttaccccctttcgacgcgggtactgcgatagtgccaccccagtctttcctactcccgactcccgaccctaacccaggttcctcggaacaggaacaccaatttactcatcccctggatgagaggaacgtcagcattttccggcccaggctaagagaagtagataagttagaatccaaattgatttatcatccccttgacgaattcgcgttggaaaaacacctctcacttgccgctcttcacacccattaatttaattcggcctctgtgttgagccccttgttgaagtgtttccctccatcgcgacgtggttggagatctaagtcaaccgactccgacgaaactaccatcatgcctccccgattatgtgatgctttctgccctgctgggtggagcaccctcgggttgagaaaaccttcttcctttttccttggactccggtcccccggtctaagccgctcggaatatgacagggttattttcacctcttcttttctactccacagtgttctatactgtggaagggtatgtgttgccccttccttcttggagaacgtgcgcggcggtctttccgtctctcgacaagcgcgcgtgcaacatacagagtaacgcgaagaaagcagttctcggtctagctctagtgcccacaagaaaacagctgtagcgaccacacaaaggcagcggaacccccctcctggtaacaggagcctctgcggccaaaagccacgtggataagatccacctttgtgtgcggtgcaaccccagcaccctggtttcttggtgacactctagtgaacccctgaatggcaatctcaagcgcctctgtagggaagccaagaatgtccaggaggtaccccttcctctcggaagggatctgacctggagacacatcacacgtgctttacacctgtgcttgtgtttaaaaattgttacagcttccccgaaccaagtggtcttggtt

SEQ ID NO: 10 - DNA, 383 bp, Hepacivirus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaac

SEQ ID NO: 11 - DNA, 12339 bp, Artificial Sequence
  CHIKV-HAV IRES
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgatgtgcaagactaccgacacggttgacggcgaaagaatgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcaacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgattgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgacctagtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtggcggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgattgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttaagactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtaaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtgattacgctttatggaggaccgaagtaataaactagttaattcctgcaggttcagggttcttaaatctgtttctctataagaacactcatttttcacgctttctgtcttctttcttccagggctctccccttgccctaggctctggccgttgcgcccggcggggtcaactccatgattagcatggagctgtaggagtctaaattggggacacagatgtttggaacgtcaccttgcagtgttaacttggctttcatgaatctctttgatcttccacaaggggtaggctacgggtgaaacctcttaggctaatacttctatgaagagatgccttggatagggtaacagcggcggatattggtgagttgttaagacaaaaaccattcaacgccggaggactgactctcatccagtggatgcattgagtggattgactgtcagggctgtctttaggcttaattccagacctctctgtgcttagggcaaacatcatttggccttaaatgggattctgtgagaggggatccctccattgacagctggactgttctttggggccttatgtggtgtttgcctctgaggtactcaggggcatttaggtttttcctcattcttaaataataatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgcgcgcggtaccacaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtaaaggggaccatcgataacgcggacctggccaaactggcctttaagcggtcatctaagtatgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaaattaataaaaatcaaatgaataccataattggcaaacggaagagatgtaggtacttaagcttcctaaaagcagccgaactcactttgagaagtaggcatagcataccgaactcttccacgattctccgaacccacagggacgtaggagatgttattttgtttttaatatttc

SEQ ID NO: 12 - DNA, 12189 bp, Artificial Sequence
  CHIKV-FGF1 IRES
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgatgtgcaagactaccgacacggttgacggcgaaagaatgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcaacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgattgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgacctagtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtggcggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgattgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttaagactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtaaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtgattacgctttatggaggaccgaagtaataaactagtgtgggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgcgcgcggtaccacaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtaaaggggaccatcgataacgcggacctggccaaactggcctttaagcggtcatctaagtatgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaaattaataaaaatcaaatgaataccataattggcaaacggaagagatgtaggtacttaagcttcctaaaagcagccgaactcactttgagaagtaggcatagcataccgaactcttccacgattctccgaacccacagggacgtaggagatgttattttgtttttaatatttc

SEQ ID NO: 13 - DNA, 584 bp, Hepatitis A virus
taattcctgcaggttcagggttcttaaatctgtttctctataagaacactcatttttcacgctttctgtcttctttcttccagggctctccccttgccctaggctctggccgttgcgcccggcggggtcaactccatgattagcatggagctgtaggagtctaaattggggacacagatgtttggaacgtcaccttgcagtgttaacttggctttcatgaatctctttgatcttccacaaggggtaggctacgggtgaaacctcttaggctaatacttctatgaagagatgccttggatagggtaacagcggcggatattggtgagttgttaagacaaaaaccattcaacgccggaggactgactctcatccagtggatgcattgagtggattgactgtcagggctgtctttaggcttaattccagacctctctgtgcttagggcaaacatcatttggccttaaatgggattctgtgagaggggatccctccattgacagctggactgttctttggggccttatgtggtgtttgcctctgaggtactcaggggcatttaggtttttcctcattcttaaataata

SEQ ID NO: 14 - DNA, 434 bp, Homo sapiens
gtgggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagcc

SEQ ID NO: 15 - DNA, 20 bp, Artificial Sequence
  primer
gaycccgactcaaccatcct

SEQ ID NO: 16 - DNA, 21 bp, Artificial Sequence
  primer
catmgggcaracgcactggta

SEQ ID NO: 17 - DNA, 25 bp, Artificial Sequence
  probe
agygcgccagcaaggaggakgatgt

SEQ ID NO: 18 - protein, 1248 aa, Artificial Sequence
  a structural peptide of CHIK-IRES
MEFIPTQTFYNRRYQPRPWTPRPTIQVIRPRPRPQRQAGQLAQLISAVNKLTMRAVPQQKPRRNRKNKKQKQKQQAPQNNTNQKKQPPKKKPAQKKKKPGRRERMCMKIENDCIFEVKHEGKVTGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLAIPVMCLLANTTFPCSQPPCTPCCYEKEPEETLRMLEDNVMRPGYYQLLQASLTCSPHRQRRSTKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKAATYQEAAIYLWNEQQPLFWLQALIPLAALIVLCNCLRLLPCCCKTLAFLAVMSVGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 19 - protein, 2474 aa, Artificial Sequence
  a non-structural peptide of CHIK-IRES
MDPVYVDIDADSAFLKALQRAYPMFEVEPRQVTPNDHANARAFSHLAIKLIEQEIDPDSTILDIGSAPARRMMSDRKYHCVCPMRSAEDPERLANYARKLASAAGKVLDRNISGKIGDLQAVMAVPDTETPTFCLHTDVSCRQRADVAIYQDVYAVHAPTSLYHQAIKGVRVAYWVGFDTTPFMYNAMAGAYPSYSTNWADEQVLKAKNIGLCSTDLTEGRRGKLSIMRGKKLKPCDRVLFSVGSTLYPESRKLLKSWHLPSVFHLKGKLSFTCRCDTVVSCEGYVVKRITMSPGLYGKTTGYAVTHHADGFLMCKTTDTVDGERMSFSVCTYVPATICDQMTGILATEVTPEDAQKLLVGLNQRIVVNGRTQRNTNTMKNYLLPVVAQAFSKWAKECRKDMEDEKLLGVRERTLTCCCLWAFKKQKTHTVYKRPDTQSIQKVQAEFDSFVVPSLWSSGLSIPLRTRIKWLLSKVPKTDLIPYSGDAREARDAEKEAEEEREAELTREALPPLQAAQEDVQVEIDVEQLEDRAGAGIIETPRGAIKVTAQPTDHVVGEYLVLSPQTVLRSQKLSLIHALAEQVKTCTHNGRAGRYAVEAYDGRVLVPSGYAISPEDFQSLSESATMVYNEREFVNRKLHHIAMHGPALNTDEESYELVRAERTEHEYVYDVDQRRCCKKEEAAGLVLVGDLTNPPYHEFAYEGLKIRPACPYKIAVIGVFGVPGSGKSAIIKNLVTRQDLVTSGKKENCQEITTDVMRQRGLEISARTVDSLLLNGCNRPVDVLYVDEAFACHSGTLLALIALVRPRQKVVLCGDPKQCGFFNMMQMKVNYNHNICTQVYHKSISRRCTLPVTAIVSSLHYEGKMRTTNEYNKPIVVDTTGSTKPDPGDLVLTCFRGWVKQLQIDYRGYEVMTAAASQGLTRKGVYAVRQKVNENPLYASTSEHVNVLLTRTEGKLVWKTLSGDPWIKTLQNPPKGNFKATIKEWEVEHASIMAGICSHQMTFDTFQNKANVCWAKSLVPILETAGIKLNDRQWSQIIQAFKEDKAYSPEVALNEICTRMYGVDLDSGLFSKPLVSVYYADNHWDNRPGGKMFGFNPEAASILERKYPFTKGKWNINKQICVTTRRIEDFNPTTNIIPANRRLPHSLVAEHRPVKGERMEWLVNKINGHHVLLVSGYNLALPTKRVTWVAPLGVRGADYTYNLELGLPATLGRYDLVVINIHTPFRIHHYQQCVDHAMKLQMLGGDSLRLLKPGGSLLIRAYGYADRTSERVICVLGRKFRSSRALKPPCVTSNTEMFFLFSNFDNGRRNFTTHVMNNQLNAAFVGQVTRAGCAPSYRVKRMDIAKNDEECVVNAANPRGLPGGGVCKAVYKKWPESFKNSATPVGTAKTVMCGTYPVIHAVGPNFSNYSESEGDRELAAAYREVAKEVTRLGVNSVAIPLLSTGVYSGGKDRLTQSLNHLFTAMDSTDADVVIYCRDKEWEKKISEAIQMRTQVELLDEHISIDCDIVRVHPDSSLAGRKGYSTTEGALYSYLEGTRFHQTAVDMAEIHTMWPKQTEANEQVCLYALGESIESIRQKCPVDDADASSPPKTVPCLCRYAMTPERVTRLRMNHVTSIIVCSSFPLPKYKIEGVQKVKCSKVMLFDHNVPSRVSPREYRSSQESAQEASTITSLTHSQFDLSVDGEILPVPSDLDADAPALEPALDDGATHTLPSTTGNLAAVSDWVMSTVPVAPPRRRRGRNLTVTCDEREGNITPMASVRFFRAELCPVVQETAETRDTAMSLQAPPSTATEPNHPPISFGASSETFPITFGDFNEGEIESLSSELLTFGDFLPGEVDDLTDSDWSTCSDTDDELRLDRAGGYIFSSDTGPGHLQQKSVRQSVLPVNTLEEVHEEKCYPPKLDEAKEQLLLKKLQESASMANRSRYQSRKVENMKAAIIQRLKRGCRLYLMSETPKVPTYRTTYPAPVYSPPINVRLSNPESAVAACNEFLARNYPTVSSYQITDEYDAYLDMVDGSESCLDRATFNPSKLRSYPKQHAYHAPSIRSAVPSPFQNTLQNVLAAATKRNCNVTQMRELPTLDSAVFNVECFKKFACNQEYWEEFAASPIRITTENLATYVTKLKGPKAAALFAKTHNLLPLQEVPMDRFTVDMKRDVKVTPGTKHTEERPKVQVIQAAEPLATAYLCGIHRELVRRLNAVLLPNVHTLFDMSAEDFDAIIAAHFKPGDTVLETDIASFDKSQDDSLALTALMLLEDLGVDHSLLDLIEAAFGEISSCHLPTGTRFKFGAMMKSGMFLTLFVNTLLNITIASRVLEDRLTKSACAAFIGDDNIIHGVVSDELMAARCATWMNMEVKIIDAVVSLKAPYFCGGFILHDTVTGTACRVADPLKRLFKLGKPLAAGDEQDEDRRRALADEVIRWQRTGLIDELEKAVYSRYEVQGISVVVMSMATFASSRSNFEKLRGPVITLYGGPK

SEQ ID NO: 20 - DNA, 565 bp, Encephalomyocarditis virus
aacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 21 - DNA, 83 bp, Artificial Sequence
  VEEV ZPC938 Wild-type
ttcagctacctgagaggggcccctataactctctacggctaacctgaatggactacgacatagtctagtccgccaagatgttc

SEQ ID NO: 22 - protein, 15 aa, Artificial Sequence
  VEEV ZPC938 Wild-type
FSILRGAPITLYGMF

SEQ ID NO: 23 - DNA, 51 bp, Artificial Sequence
  VEEV ZPC938 Mutant
ttcagctacctgagaggggccccgattacgttgtatggatgataaatgttc

SEQ ID NO: 24 - DNA, 83 bp, Artificial Sequence
  VEEV 68U201 Wild-type
ttcagttgtctaagaggggcttctatatccctctacggctaacctgaatggactgcgacgtagtcaagtccgccgaaatgttt

SEQ ID NO: 25 - protein, 15 aa, Artificial Sequence
  VEEV 68U201 Wild-type
FSCLRGASISLYGMF

SEQ ID NO: 26 - DNA, 57 bp, Artificial Sequence
  VEEV 68U201 Mutant
ttcagttgtctaagaggggcttccataagtctgtatggatgataaatcgatatgttt

SEQ ID NO: 27 - DNA, 92 bp, WEEV Imp181 Wild-type
ttcaagagcataagagggagcccaatcaccctctacggctgacctaaataggtgacgtagtagacacgcacctacccatcgccataatgttt

SEQ ID NO: 28 - protein, 15 aa, Artificial Sequence
  WEEV Imp181 Wild-type
FKSIRGSLITLYGMF

SEQ ID NO: 29 - DNA, 51 bp, Artificial Sequence
  WEEV Imp181 Mutant
ttcaagagcataagagggagcccgattacgttgtatggataataaatgttt

SEQ ID NO: 30 - DNA, 105 bp, Artificial Sequence
  EEEV FL93-939 Wild-type
cacataagaggtcaccccataaccctctacggctgacctaaataggttgtgcattagtacctaacctatttatattatattgctatctaaatatcagagatgttc

SEQ ID NO: 31 - protein, 13 aa, Artificial Sequence
  EEEV FL93-939 Wild-type
HIRGHPITLYGMF

SEQ ID NO: 32 - DNA, 52 bp, Artificial Sequence
  EEEV FL93-939 Mutant
cacataagaggtcacccgattacgttgtatggataataaaacgttaatgttc

SEQ ID NO: 33 - DNA, 117 bp, Artificial Sequence
  CHIKV LaReunion Wild-type
aagctcagaggacccgtcataactttgtacggcggtcctaaataggtacgcactacagctacctattttgcagaagccgacagcaagtatctaaaacactaatcagctacaatggag

SEQ ID NO: 34 - protein, 16 aa, Artificial Sequence
  CHIKV LaReunion Wild-type
KLRGPVITLYGGPKME

SEQ ID NO: 35 - DNA, 60 bp, Artificial Sequence
  CHIKV LaReunion Mutant
aagctcagaggacccgtgattacgctttatggaggaccgaagtaataaactagtatggag